(19) United States
(12) Patent Application Publication
(10) Pub. No.: US 2010/0167291 A1
(43) Pub. Date: Jul. 1, 2010

(54) ASSESSING EXPRESSION OF ENDOGENOUS AND EXOGENOUS GENES

(75) Inventors: Martin Rosenberg, Madison, WI (US); Douglas Storts, Verona, WI (US)

Correspondence Address: Michael Best & Friedrich LLP, 100 East Wisconsin Avenue, Suite 3300, Milwaukee, WI, 53202, US

(73) Assignee: PROMEGA CORPORATION, Madison, WI (US)

(21) Appl. No.: 12/539,436

(22) Filed: Aug. 11, 2009

Related U.S. Application Data
(60) Provisional application No. 61/096,536, filed on Sep. 12, 2008.

(51) Int. Cl. C12Q 1/68

(52) U.S. Cl. 435/6

(57) ABSTRACT

The present invention relates to compositions and methods to assess gene expression in cells. In particular, the present invention provides compositions and methods to assess expression from an exogenous gene and an endogenous version of the same gene in induced pluripotent stem cells.

The present application claims priority to U.S. Provisional Patent Application Ser. No. 61/096,536, filed Sep. 12, 2008, the disclosure of which is herein incorporated by reference in its entirety.

### FIELD OF THE INVENTION

The present invention relates to compositions and methods to assess gene expression in cells. In particular, the present invention provides compositions and methods to assess expression from an exogenous gene and an endogenous version of the same gene in induced pluripotent stem cells.

### BACKGROUND OF THE INVENTION

The formation of tissues and organs occurs naturally during prenatal development. The development of multicellular organisms follows pre-determined molecular and cellular pathways culminating in the formation of entities composed of billions of cells with defined functions. Cellular development is accomplished through cellular proliferation, lineage-commitment, and lineage-progression, resulting in the formation of differentiated cell types. This process begins with the totipotent zygote and continues throughout the life of the individual. As development proceeds from the totipotent zygote, cells proliferate and segregate by lineage-commitment into the pluripotent primary germ layers, ectoderm, mesoderm, and endoderm. Further segregation of these germ layers through progressive lineage-commitment into progenitor (multipotent, tripotent, bipotent and eventually unipotent) lineages further defines the differentiation pathways of the cells and their ultimate function.

Development proceeds from the fertilized egg, to formation of a blastula and then a gastrula. Gastrulation is the process by which the bilaminar embryonic disc is converted into a trilaminar embryonic disc. Gastrulation is the beginning of morphogenesis or development of the body form gastrulation begins with the formation of the primitive streak on the surface of the epiblast of the embryonic disk. Formation of the primitive streak, germ layers, and notochord are the important processes occurring during gastrulation. Each of the three germ layers—ectoderm, endoderm, and mesoderm—gives rise to specific tissues and organs. The organization of the embryo into three layers roughly corresponds to the organization of the adult, with gut on the inside, epidermis on the outside, and connective tissue in between.

While a majority of the cells progress through the sequence of development and differentiation, a few cells leave this pathway to become reserve stem cells that provide for the continual maintenance and repair of the organism. Reserve stem cells include progenitor stem cells and pluripotent stem cells. Progenitor cells (e.g., precursor stem cells, immediate stem cells, and forming or -blast cells, e.g., myoblasts, adipoblasts, chondroblasts, etc.) are lineage-committed. Unipotent stem cells will form tissues restricted to a single lineage (such as the myogenic, fibrogenic, adipogenic, chondrogenic, osteogenic lineages, etc.). Bipotent stem cells will form tissues belonging to two lineages (such as the chondro-osteogenic, adipo-fibroblastic lineages, etc.). Tripotent stem cells will form tissues belonging to three lineages (such as chondro-osteoadipogenic lineage, etc.). Multipotent stem cells will form multiple cell types within a lineage (such as the hematopoietic lineage). Progenitor stem cells will form tissues limited to their lineage, regardless of the inductive agent that may be added to the medium. They can remain quiescent. Lineage-committed progenitor cells are capable of self-replication but have a limited life-span (approximately 50-70 cell doublings) before programmed cell senescence occurs. They can also be stimulated by various growth factors to proliferate. If activated to differentiate, these cells require progression factors (e.g., insulin, insulin-like growth factor-I, and insulin-like growth factor-II) to stimulate phenotypic expression.

In contrast, pluripotent cells are lineage-uncommitted, i.e., they are not committed to any particular tissue lineage. They can remain quiescent. They can also be stimulated by growth factors to proliferate. If activated to proliferate, pluripotent cells are capable of extended self-renewal as long as they remain lineage-uncommitted. Pluripotent cells have the ability to generate various lineage-committed progenitor cells from a single clone at any time during their life span. This lineage-commitment process necessitates the use of either general (e.g., dexamethasone) or lineage-specific (e.g., bone morphogenetic protein-2, muscle morphogenetic protein, etc.) commitment induction agents. Once pluripotent cells are induced to commit to a particular tissue lineage, they assume the characteristics of lineage-specific progenitor cells. They can remain quiescent or they can proliferate, under the influence of specific inductive agents.

Embryonic stem cells are uncommitted, totipotent cells isolated from embryonic tissue. When injected into embryos, they can give rise to all somatic lineages as well as functional gametes. Upon differentiation these cells express a wide variety of cell types, derived from ectodermal, mesoderm, and endodermal embryonic germ layers. Embryonic stem (ES) cells have been isolated from the blastocyst, inner cell mass or gonadal ridges of mouse, rabbit, rat, pig, sheep, primate and human embryos.

Induced pluripotent stem cells (iPSCs) are a type of pluripotent stem cell artificially derived from a non-pluripotent cell, typically an adult somatic cell, by inducing a “forced” expression of certain genes. IPSCs are believed to share many characteristics with natural pluripotent stem cells, such as embryonic stem cells, including the expression of certain stem cell genes and proteins, chromatin methylation patterns, doubling time, embryoid body formation, teratoma formation, viable chimera formation, and potency and differentiability. The full extent of their relation to natural pluripotent stem cells is still being assessed. IPSCs were first produced in 2006 from mouse cells (Takahashi et al., Cell 2006, 126:663-676, herein incorporated by reference in its entirety) and in 2007 from human cells (Vodyanik et al., Science 2007, 318(5858):1917-20, herein incorporated by reference in its entirety).

IPSCs are typically derived by transfection of certain stem cell-associated genes into non-pluripotent cells, such as adult fibroblasts. Transfected genes include the master transcriptional regulators (e.g. Oct-3/4 and Sox2) as well as other genes that enhance the efficiency of induction. The exogenous genes induce expression of endogenous genes causing the reprogramming of the somatic cells into pluripotent stem cells. Once small numbers of transfected cells begin to become morphologically and biochemically similar to pluripotent stem cells, they are typically isolated through morphological selection, doubling time, or through a reporter gene and antibiotic selection. There is a need in the art to better understand iPSCs and the roles of exogenous and endogenous genes in their creation, maintenance, and use as research materials (e.g., for basic research, drug screening, and the like) or therapeutic materials.

### SUMMARY

The present invention provides compositions and methods to assess gene expression in cells. In particular, the present invention provides compositions (e.g., reagents, kits, reaction mixtures, and the like) and methods to assess expression from an exogenous gene and an endogenous version of the same gene in induced pluripotent stem cells. These compositions and methods find use in a wide variety of applications including, but not limited to, empirical investigation of iPSCs, drug screening, cell therapies, and the like.

The biological materials that are used to generate iPSCs are native genes found in the cells that are to be de-differentiated. For example, exogenous genetic material may be introduced into a cell to activate a de-differentiation process. This exogenous genetic material typically encodes for transcription factors that may be endogenously expressed in the treated cell. Thus, one problem in working with iPSCs and understanding their biology is the inability to determine whether an expression product (e.g., mRNA, protein) in the cells is the result of endogenous gene expression or the result of exogenous administration. The compositions and methods of the present invention provide the ability to differentiate between endogenous and exogenous expression. For example, in some embodiments, the present invention provides synthetic nucleic acid molecules that encode a de-differentiation factor, wherein the synthetic nucleic acid molecule differs in sequences from the corresponding endogenous gene found in a target cell. In some embodiments, the synthetic nucleic acid encodes for a native protein sequence, but contains differences in codon usage or other sequences so as to exhibit a nucleic acid sequence that differs from the endogenous gene. In some embodiments, the synthetic sequence encodes for a different amino acid sequence, although preferably one that maintains de-differentiation activity. Differences in the nucleic acid or amino acid sequence are identified using any of a wide variety of approaches to distinguish endogenous from exogenous expression. The present invention further provides reagents (e.g., primers, probes, and the like) for readily differentiating endogenous versus exogenous gene expression of a de-differentiating factor (e.g., primers or probes specific for the synthetic sequence relative to the native sequence).

The compositions and methods of the present invention find use, for example, with stem cells, derived from non-embryonic animal cells or tissue, capable of self regeneration and capable of differentiation to cells of endodermal, ectodermal and mesodermal lineages. The compositions and methods of the present invention find particular use with pluripotent embryonic-like stem cells, derived from postnatal (e.g., adult) animal cells or tissue, capable of self regeneration and capable of differentiation to cells of endodermal, ectodermal and mesodermal lineages. The pluripotent embryonic-like stem cells may be isolated from non-human cells or human cells. For example, the pluripotent embryonic-like stem cells may be isolated from the non-embryonic tissue selected from the group of muscle, dermis, fat, tendon, ligament, perichondrium, periosteum, heart, aorta, endocardium, myocardium, epicardium, large arteries and veins, granulation tissue, peripheral nerves, peripheral ganglia, spinal cord, dura, leptomeninges, trachea, esophagus, stomach, small intestine, large intestine, liver, spleen, pancreas, parietal peritoneum, visceral peritoneum, parietal pleura, visceral pleura, urinary bladder, gall bladder, kidney, associated connective tissues or bone marrow, among others.

In some embodiments, the present invention provides pluripotent embryonic-like stem cells, or populations of such cells, which have been transformed or transfected and thereby contain and can express an exogenous non-native gene or protein of interest. Thus, this invention includes pluripotent embryonic-like stem cells genetically engineered to express an exogenous gene or protein of interest. In as much as such genetically engineered stem cells can then undergo lineage-commitment, the present invention further encompasses lineage-committed cells, which are derived from a genetically engineered pluripotent embryonic-like stem cell, and which express an exogenous gene or protein of interest. The lineage-committed cells may be endodermal, ectodermal or mesodermal lineage-committed cells and may be pluripotent, such as a pluripotent mesenchymal stem cell, or progenitor cells, such as an adipogenic or a myogenic cell.

In some embodiments, the present invention provides methods of producing a genetically engineered pluripotent embryonic-like stem cell comprising the steps of: transfecting a cell or population of cells with a nucleic acid molecule encoding for a de-differentiation gene having a non-native sequence; selecting for expression of the dedifferentiation gene; and culturing the selected stem cells. In some embodiments, the method further comprises the step of detecting the presence of, absence of, or amount of expression of said non-native de-differentiation gene. In some embodiments, the method further comprises the step of detecting the presence of, absence of, or amount of endogenous expression of said de-differentiation gene in said cell or cells.

The present invention is not limited by the manner in which the synthetic normative sequence differs from the endogenous sequence. Any difference that can be differentially detected may be used. In some embodiments, the detectable property is a difference in RNA sequence of the exogenous gene and the endogenous version of the same gene. In some embodiments the difference in RNA sequence occurs in the wobble position of RNA codons. For example, where the endogenous gene uses the codon CGT to encode arginine, the non-native sequence may use CGC, CGA, or CGG. In some embodiments, the difference in RNA sequence occurs in an untranslated region. One or more (e.g., 2, 3, 4, 5, 6, 7, 8, 9, 10, or more) nucleotides may be different between the synthetic and endogenous sequences.

In some embodiments, the methods for detecting expression of exogenous and endogenous genes in cells, stem cells, adult stem cells, embryonic stem cells, pluripotent stem cells, and induced pluripotent stem cells, involve the steps of extracting RNA from cells, amplifying a fraction of the extracted RNA or the corresponding cDNA, wherein the fraction comprises RNA of exogenous and endogenous genes, and detecting the amplified product of RNA of exogenous and endogenous genes or their cDNA. Probes, primers, antibodies, or other reagents specific for the endogenous and/or exogenous genes may be employed. For example, in some embodiments, primers used to amplify the RNA or cDNA contain a base at their 3′ end that is complementary to the synthetic sequence, but not complementary to the endogenous sequence, thus only being extendable when hybridized to the synthetic sequence, if present in the sample. In some embodiments, the primers amplify both the endogenous and exogenous sequences, but generate amplicons that differ in size or sequence so as to be readily differentiated. In other embodiments, probes specific for the either the synthetic or endogenous sequence are used. Skilled artisans will understand a wide variety of techniques that are available for distinguishing between the sequences.

In some embodiments, where RNA is extracted analyzed, RNA or the corresponding cDNA is amplified using an amplification method including, but not limited to, reverse transcriptase polymerase chain reaction (PCR) (e.g., PLEXOR, Promega, Madison, Wis., see e.g., U.S. Pat. Publ. No. 20020150900, herein incorporated by reference in its entirety; TAQMAN, etc.); ligase chain reaction; Q-beta replication; transcription-based amplification; isothermal nucleic acid sequence based amplification techniques; micro array analysis; nucleic acid sequencing; and any combination or variation thereof.

The invention further provides kits containing one or more components useful, necessary, or sufficient for carrying out methods described herein. Kit components may include, but are not limited to, primers, probes, enzymes, buffers, cells, control reagents or cells (e.g., positive controls, negative controls), dyes, hardware (e.g., microcentrifuge tubes and the like), detection equipments, software for data collection, analysis, or other purposes, instructions for practicing the methods, and the like. The kit may be provided as a single container housing the various components or may be provided in two or more different containers.

In some embodiments of the present invention, the de-differentiation factor is a transcription factor. In some embodiments the de-differentiation factor is one or more of: Oct1, Oct3, Oct4, Oct3/4, Oct6, SoxI, Sox2, Sox3, Sox15, Sox18, Nanog, C-Myc, N-Myc, L-Myc, Klf1, Klf2, Klf4, Klf5, LIN28, and Fbx15.

As used herein, the term “gene transfer system” refers to any means of delivering a composition comprising a nucleic acid sequence to a cell or tissue. For example, gene transfer systems include, but are not limited to, vectors (e.g., retroviral, adenoviral, adeno-associated viral, and other nucleic acid-based delivery systems), microinjection of naked nucleic acid, polymer-based delivery systems (e.g., liposome-based and metallic particle-based systems), biolistic injection, and the like. As used herein, the term “viral gene transfer system” refers to gene transfer systems comprising viral elements (e.g., intact viruses, modified viruses and viral components such as nucleic acids or proteins) to facilitate delivery of the sample to a desired cell or tissue. As used herein, the term “adenovirus gene transfer system” refers to gene transfer systems comprising intact or altered viruses belonging to the family Adenoviridae.

As used herein, the term “site-specific recombination target sequences” refers to nucleic acid sequences that provide recognition sequences for recombination factors and the location where recombination takes place.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N-6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, I-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N 6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

When used in reference to a single-stranded nucleic acid sequence, the term “substantially homologous” refers to any probe that can hybridize (i.e., it is the complement of) the single-stranded nucleic acid sequence under conditions of low stringency.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under “medium stringency conditions,” a nucleic acid sequence of interest will hybridize only to its exact complement, sequences with single base mismatches, and closely relation sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

“High stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 0.1×SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 1.0×SSPE, 1.0% SDS at 42° C. when a probe of about 500 nucleotides in length is employed.

“Low stringency conditions” comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.1% SDS, 5×Denhardt's reagent [50×Denhardt's contains per 500 ml: 5 g Ficoll (Type 400, Pharamcia), 5 g BSA (Fraction V; Sigma)] and 100 μg/ml denatured salmon sperm DNA followed by washing in a solution comprising 5×SSPE, 0.1% SDS at 42° C. when a probe of about 500 nucleotides in length is employed.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.) (see definition above for “stringency”).

As used herein, the term “amplification oligonucleotide” refers to an oligonucleotide that hybridizes to a target nucleic acid, or its complement, and participates in a nucleic acid amplification reaction. An example of an amplification oligonucleotide is a “primer” that hybridizes to a template nucleic acid and contains a 3′ OH end that is extended by a polymerase in an amplification process. Another example of an amplification oligonucleotide is an oligonucleotide that is not extended by a polymerase (e.g., because it has a 3′ blocked end) but participates in or facilitates amplification. Amplification oligonucleotides may optionally include modified nucleotides or analogs, or additional nucleotides that participate in an amplification reaction but are not complementary to or contained in the target nucleic acid. Amplification oligonucleotides may contain a sequence that is not complementary to the target or template sequence. For example, the 5′ region of a primer may include a promoter sequence that is non-complementary to the target nucleic acid (referred to as a “promoter-primer”). Those skilled in the art will understand that an amplification oligonucleotide that functions as a primer may be modified to include a 5′ promoter sequence, and thus function as a promoter-primer. Similarly, a promoter-primer may be modified by removal of, or synthesis without, a promoter sequence and still function as a primer. A 3′ blocked amplification oligonucleotide may provide a promoter sequence and serve as a template for polymerization (referred to as a “promoter-provider”).

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer should be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, that is capable of hybridizing to at least a portion of another oligonucleotide of interest. A probe may be single-stranded or double-stranded. Probes are useful in the detection, identification and isolation of particular gene sequences. It is contemplated that any probe used in the present invention will be labeled with any “reporter molecule, n so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

“Lineage-commitment” refers to the process by which individual cells commit to subsequent and particular stages of differentiation during the developmental sequence leading to the formation of a life form.

The term “lineage-uncommitted” refers to a characteristic of cell(s) whereby the particular cell(s) are not committed to any next subsequent stage of differentiation (e.g., germ layer lineage or cell type) of the developmental sequence.

The term “lineage-committed” refers to a characteristic of cell(s) whereby the particular cell(s) are committed to a particular next subsequent stage of differentiation (e.g., germ layer lineage or cell type) of the developmental sequence. Lineage-committed cells, for instance, can include those cells which can give rise to progeny limited to a single lineage within a germ layers, e.g., liver, thyroid (endoderm), muscle, bone (mesoderm), neuronal, melanocyte, epidermal (ectoderm), etc.

A “clone” or “clonal population” is a population of cells derived from a single cell or common ancestor by mitosis. A “cell line” is a clone of a primary cell that is capable of stable growth in vitro for many generations.

A cell has been “transformed” or “transfected” by exogenous or heterologous DNA when such DNA has been introduced inside the cell. The transforming or transfecting DNA may or may not be integrated (covalently linked) into chromosomal DNA making up the genome of the cell. In prokaryotes, yeast, and mammalian cells for example, the transforming or transfecting DNA may be maintained on an episomal element such as a plasmid. With respect to eukaryotic cells, a stably transformed or transfected cell is one in which the transforming or transfecting DNA has become integrated into a chromosome so that it is inherited by daughter cells through chromosome replication. This stability is demonstrated by the ability of the eukaryotic cell to establish cell lines or clones comprised of a population of daughter cells containing the transforming or transfecting DNA.

### BRIEF DESCRIPTION OF THE DRAWINGS

The foregoing summary and detailed description is better understood when read in conjunction with the accompanying drawings which are included by way of example and not by way of limitation.

FIG. 1 shows the sequences of exemplary (a) endogenous and (b) exogenous Nanog genes. The locations and amino acid sequences of the endogenous forward primer binding site (ENDO-FPBS), endogenous reverse primer binding site (ENDO-RPBS), exogenous forward primer binding site (EXO-FPBS), and exogenous reverse primer binding site (EXO-RPBS) are indicated. Differences in nucleotide sequence between the exogenous and endogenous genes are highlighted in the exogenous sequence. The figure demonstrates that the two genes have different nucleotide sequences, but code for the same amino acid sequence. The figure illustrates that the amplicon produced by the exogenous primer binding sites is significantly larger than that produced by the endogenous primer binding sites.

FIG. 2 shows the sequences of exemplary (a) endogenous and (b) exogenous primers for use with the endogenous and exogenous Nanog genes of FIG. 1. Differences in nucleotide sequence between the exogenous and endogenous genes are highlighted in the exogenous sequence.

### DETAILED DESCRIPTION OF EMBODIMENTS

Induced pluripotent stem cells (iPSCs) are a type of pluripotent stem cell derived (e.g. artificially derived) from a non-pluripotent cell, typically an adult somatic cell, by inducing a “forced” expression of certain genes. IPSCs are believed to share many characteristics with natural pluripotent stem cells, such as embryonic stem cells, such as the expression of certain stem cell genes and proteins, chromatin methylation patterns, doubling time, embryoid body formation, teratoma formation, viable chimera formation, and potency and differentiability. The full extent of their relation to natural pluripotent stem cells is still being assessed.

IPSCs are typically derived by transfection of certain stem cell-associated genes into non-pluripotent cells, such as adult fibroblasts. Transfected genes include the master transcriptional regulators (e.g. Oct-3/4 and Sox2) as well as other genes that enhance the efficiency of induction. The exogenous genes induce expression of endogenous versions of the same genes. Once small numbers of transfected cells begin to become morphologically and biochemically similar to pluripotent stem cells, they are typically isolated through morphological selection, doubling time, or through a reporter gene and antibiotic selection.

The present invention provides compositions and methods to assess the expression of endogenous and exogenous genes in induced pluripotent stem cells. Induced pluripotent stem cells are generated from somatic cells by introducing exogenous versions of transcription factors known to induce pluripotency (e.g. Oct4, SOX2, Nanog, etc) to induce the expression of endogenous genes. Monitoring mRNA expression of both endogenous and exogenous genes is useful for understanding and monitoring that the induction to pluripotency and to monitor and understand the cell's later manipulations, such as subsequent differentiation. Compositions and methods of the present invention provide synthetic genes that express a transcription factor similar or identical to the endogenous gene coding for one of the endogenous inducing factors.

The synthetic genes encode for RNA sequences that differ from the endogenous gene. The difference in mRNA sequence between the endogenous and exogenous versions of the gene allows for the use of endogenous- and exogenous-specific reagents for differentiating between endogenous and exogenous expression. The synthetic gene, in the appropriate gene transfer system is used to induce somatic cells into an undifferentiated, pluripotent state.

The present invention is not limited by the manner in which the synthetic normative sequence differs from the endogenous sequence. Any difference that can be differentially detected may be used. In some embodiments, the detectable property is a difference in RNA sequence of the exogenous gene and the endogenous version of the same gene. In some embodiments the difference in RNA sequence occurs in the wobble position of RNA codons. For example, where the endogenous gene uses the codon CGT to encode arginine, the non-native sequence may use CGC, CGA, or CGG. In some embodiments, the difference in RNA sequence occurs in an untranslated region. One or more (e.g., 2, 3, 4, 5, 6, 7, 8, 9, 10, 20, 50, or more) nucleotides may be different between the exogenous (e.g. transfected, synthetic, etc.) and endogenous sequences. In some embodiments, exogenous and endogenous sequences may comprise one or more regions of differentiable sequence (e.g. 1, 2, 3, 4, 5, 6, 7, 8, 9, 10, or more). In some embodiments a differentiable region comprises one or more different nucleotides between exogenous and endogenous sequences. In some embodiments, differentiable regions provide primer or probe binding regions. In some embodiments, one or more primers and/or probes are configured to bind to a differentiable region in either the endogenous sequence or the exogenous sequence, but not both. In some embodiments, binding of primers or probes to either an exogenous sequence or an endogenous sequence, but not both, provides a mechanism for differentiating between endogenous and exogenous sequences. In some embodiments, despite containing one or more differentiable regions, exogenous and endogenous sequences code for proteins with identical amino acid sequence.

The compositions and methods of the present find use in a variety of applications including, but not limited to, conducting empirical research to understand iPSC biology; monitoring cell status in response to de-differentiation, differentiation, or drug treatment regiments; quality control/quality assessment of cells; assessment of cells prior to or after culturing or transplantation; and the like. The detection and analysis of the endogenous versus the synthetic, non-native gene expression products may be conducted using any of variety of techniques. Several non-limiting example are described below in more detail.

In accordance with the present invention there may be employed conventional molecular biology, microbiology, and recombinant DNA techniques within the skill of the art. Such techniques are explained in the literature. See, e.g., Sambrook et al., “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984): “Nucleic Acid Hybridization” [B. D. Hames & S. J. Higgins eds. (1985)]; “Transcription And Translation” [B. D. PHames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

### A. Sample

Any sample containing, or suspected of containing, the exogenous or endogenous genes of interest may be tested according to the methods of the present invention. By way of non-limiting examples, the sample may be isolated nucleic acid molecules, cell lysates, cells, tissues, or fluids.

### B. DNA and RNA Detection

The DNA (e.g., cDNA) or RNA (e.g., mRNA) may be detected using a variety of nucleic acid techniques known to those of ordinary skill in the art, including but not limited to nucleic acid sequencing, nucleic acid hybridization, and nucleic acid amplification.

### 1. Sequencing

Illustrative non-limiting examples of nucleic acid sequencing techniques include, but are not limited to, chain terminator (Sanger) sequencing and dye terminator sequencing. Those of ordinary skill in the art will recognize that for practical reasons (e.g. because RNA is less stable in the cell and more prone to nuclease attack experimentally) RNA is often (e.g. usually) reverse transcribed to DNA before sequencing.

Chain termination sequencing uses sequence-specific termination of a DNA synthesis reaction using modified nucleotide substrates. Extension is initiated at a specific site on the template DNA by using a short radioactive, or other labeled, oligonucleotide primer complementary to the template at that region. The oligonucleotide primer is extended using a DNA polymerase, standard four deoxynucleotide bases, and a low concentration of one chain terminating nucleotide, most commonly a di-deoxynucleotide. This reaction is repeated in four separate tubes with each of the bases taking turns as the di-deoxynucleotide. Limited incorporation of the chain terminating nucleotide by the DNA polymerase results in a series of related DNA fragments that are terminated only at positions where that particular di-deoxynucleotide is used. For each reaction tube, the fragments are size-separated by electrophoresis in a slab polyacrylamide gel or a capillary tube filled with a viscous polymer. The sequence is determined by reading which lane produces a visualized mark from the labeled primer as you scan from the top of the gel to the bottom.

Dye terminator sequencing alternatively labels the terminators. Complete sequencing can be performed in a single reaction by labeling each of the di-deoxynucleotide chain-terminators with a separate fluorescent dye, which fluoresces at a different wavelength.

### 2. Hybridization

Illustrative non-limiting examples of nucleic acid hybridization techniques include, but are not limited to, in situ hybridization (ISH), microarray, and Southern or Northern blot.

In situ hybridization (ISH) is a type of hybridization that uses a labeled complementary DNA or RNA strand as a probe to localize a specific DNA or RNA sequence in a portion or section of tissue (in situ), or, if the tissue is small enough, the entire tissue (whole mount ISH). DNA ISH can be used to determine the structure of chromosomes. RNA ISH is used to measure and localize mRNAs and other transcripts within tissue sections or whole mounts. Sample cells and tissues are usually treated to fix the target transcripts in place and to increase access of the probe. The probe hybridizes to the target sequence at elevated temperature, and then the excess probe is washed away. The probe that was labeled with either radio-, fluorescent- or antigen-labeled bases is localized and quantitated in the tissue using either autoradiography, fluorescence microscopy or immunohistochemistry, respectively. ISH can also use two or more probes, labeled with radioactivity or the other non-radioactive labels, to simultaneously detect two or more transcripts.

Different kinds of biological assays are called micro arrays including, but not limited to: DNA micro arrays (e.g., cDNA microarrays and oligonucleotide microarrays); protein microarrays; tissue microarrays; transfection or cell microarrays; chemical compound microarrays; and, antibody microarrays. A DNA microarray, commonly known as gene chip, DNA chip, or biochip, is a collection of microscopic DNA spots attached to a solid surface (e.g., glass, plastic or silicon chip) forming an array for the purpose of expression profiling or monitoring expression levels for thousands of genes simultaneously. The affixed DNA segments are known as probes, thousands of which can be used in a single DNA microarray. Microarrays can be fabricated using a variety of technologies, including but not limiting: printing with fine-pointed pins onto glass slides; photolithography using pre-made masks; photolithography using dynamic micromirror devices; ink-jet printing; or, electrochemistry on microelectrode arrays.

Southern and Northern blotting is used to detect specific DNA or RNA sequences, respectively. DNA or RNA extracted from a sample is fragmented, electrophoretically separated on a matrix gel, and transferred to a membrane filter. The filter bound DNA or RNA is subject to hybridization with a labeled probe complementary to the sequence of interest. Hybridized probe bound to the filter is detected. A variant of the procedure is the reverse Northern blot, in which the substrate nucleic acid that is affixed to the membrane is a collection of isolated DNA fragments and the probe is RNA extracted from a tissue and labeled.

### 3. Amplification

Chromosomal rearrangements of genomic DNA and chimeric mRNA may be amplified prior to or simultaneous with detection. Illustrative non-limiting examples of nucleic acid amplification techniques include, but are not limited to, polymerase chain reaction (PCR), reverse transcription polymerase chain reaction (RT-PCR), transcription-mediated amplification (TMA), ligase chain reaction (LCR), strand displacement amplification (SDA), and nucleic acid sequence based amplification (NASBA). Those of ordinary skill in the art will recognize that certain amplification techniques (e.g., PCR) may involve having RNA be reversed transcribed to DNA prior to amplification (e.g., RT-PCR), whereas other amplification techniques directly amplify RNA (e.g., TMA and NASBA).

The polymerase chain reaction (U.S. Pat. Nos. 4,683,195, 4,683,202, 4,800,159 and 4,965,188, each of which is herein incorporated by reference in its entirety), commonly referred to as PCR, uses multiple cycles of denaturation, annealing of primer pairs to opposite strands, and primer extension to exponentially increase copy numbers of a target nucleic acid sequence. In a variation called RT-PCR, reverse transcriptase (RT) is used to make a complementary DNA (cDNA) from mRNA, and the cDNA is then amplified by PCR to produce multiple copies of DNA. For other various permutations of PCR see, e.g., U.S. Pat. Nos. 4,683,195, 4,683,202 and 4,800,159; Mullis et al., Meth. Enzymol. 155: 335 (1987); and, Murakawa et al., DNA 7: 287 (1988), each of which is herein incorporated by reference in its entirety. A wide variety of amplification approaches use PCR as part of the detection process. For example, the PLEXOR technology (Promega), which employs non-natural nucleotide sequences, may be used (see, U.S. Pat. Publ. No. 20020150900).

Transcription mediated amplification (U.S. Pat. Nos. 5,480,784 and 5,399,491, each of which is herein incorporated by reference in its entirety), commonly referred to as TMA, synthesizes multiple copies of a target nucleic acid sequence autocatalytically under conditions of substantially constant temperature, ionic strength, and pH in which multiple RNA copies of the target sequence autocatalytically generate additional copies. See, e.g., U.S. Pat. Nos. 5,399,491 and 5,824,518, each of which is herein incorporated by reference in its entirety. In a variation described in U.S. Publ. No. 20060046265 (herein incorporated by reference in its entirety), TMA optionally incorporates the use of blocking moieties, terminating moieties, and other modifying moieties to improve TMA process sensitivity and accuracy.

The ligase chain reaction (Weiss, R., Science 254: 1292 (1991), herein incorporated by reference in its entirety), commonly referred to as LCR, uses two sets of complementary DNA oligonucleotides that hybridize to adjacent regions of the target nucleic acid. The DNA oligonucleotides are covalently linked by a DNA ligase in repeated cycles of thermal denaturation, hybridization and ligation to produce a detectable double-stranded ligated oligonucleotide product.

Strand displacement amplification (Walker, G. et al., Proc. Natl. Acad. Sci. USA 25 89: 392-396 (1992); U.S. Pat. Nos. 5,270,184 and 5,455,166, each of which is herein incorporated by reference in its entirety), commonly referred to as SDA, uses cycles of annealing pairs of primer sequences to opposite strands of a target sequence, primer extension in the presence of a dNTPaS to produce a duplex hemiphosphorothioated primer extension product, endonuclease-mediated nicking of a hemimodified restriction endonuclease recognition site, and polymerase-mediated primer extension from the 3′ end of the nick to displace an existing strand and produce a strand for the next round of primer annealing, nicking and strand displacement, resulting in geometric amplification of product. Thermophilic SDA (tSDA) uses thermophilic endonucleases and polymerases at higher temperatures in essentially the same method (EP Pat. No. 0 684315).

Other amplification methods include, for example: nucleic acid sequence based amplification (U.S. Pat. No. 5,130,238, herein incorporated by reference in its entirety), commonly referred to as NASBA; one that uses an RNA replicase to amplify the probe molecule itself (Lizardi et al., Bio Technol. 6: 1197 (1988), herein incorporated by reference in its entirety), commonly referred to as Qβ replicase; a transcription based amplification method (Kwoh et al., Proc. Natl. Acad. Sci. USA 86:1173 (1989); and, self-sustained sequence replication (Guatelli et al., Proc. Natl. Acad. Sci. USA 87: 1874 (1990), each of which is herein incorporated by reference in its entirety). For further discussion of known amplification methods see Persing, David H., “In Vitro Nucleic Acid Amplification Techniques” in Diagnostic Medical Microbiology: Principles and Applications (Persing et al., Eds.), pp. 51-87 (American Society for Microbiology, Washington, D.C. (1993).

### 4. Detection Methods

Non-amplified or amplified nucleic acids can be detected by any conventional means. For example, the sequences can be detected by hybridization with a detectably labeled probe and measurement of the resulting hybrids. Illustrative non-limiting examples of detection methods are described below.

One illustrative detection method, the Hybridization Protection Assay (HPA) involves hybridizing a chemiluminescent oligonucleotide probe (e.g., an acridinium ester-labeled (AE) probe) to the target sequence, selectively hydrolyzing the chemiluminescent label present on unhybridized probe, and measuring the chemiluminescence produced from the remaining probe in a luminometer. See, e.g., U.S. Pat. No. 5,283,174 and Norman C. Nelson et al., Nonisotopic Probing, Blotting, and Sequencing, ch. 17 (Larry J. Kricka ed., 2d ed. 1995, each of which is herein incorporated by reference in its entirety).

Another illustrative detection method provides for quantitative evaluation of the amplification process in real-time. Evaluation of an amplification process in “real-time” involves determining the amount of amplicon in the reaction mixture either continuously or periodically during the amplification reaction, and using the determined values to calculate the amount of target sequence initially present in the sample. A variety of methods for determining the amount of initial target sequence present in a sample based on real-time amplification are well known in the art. These include methods disclosed in U.S. Pat. Nos. 6,303,305 and 6,541,205, each of which is herein incorporated by reference in its entirety. Another method for determining the quantity of target sequence initially present in a sample, but which is not based on a real-time amplification, is disclosed in U.S. Pat. No. 5,710,029, herein incorporated by reference in its entirety.

Amplification products may be detected in real-time through the use of various self-hybridizing probes, most of which have a stem-loop structure. Such self-hybridizing probes are labeled so that they emit differently detectable signals, depending on whether the probes are in a self-hybridized state or an altered state through hybridization to a target sequence. By way of non-limiting example, “molecular torches” are a type of self-hybridizing probe that includes distinct regions of self-complementarity (referred to as “the target binding domain” and “the target closing domain”) which are connected by a joining region (e.g., non-nucleotide linker) and which hybridize to each other under predetermined hybridization assay conditions. In a preferred embodiment, molecular torches contain single-stranded base regions in the target binding domain that are from 1 to about 20 bases in length and are accessible for hybridization to a target sequence present in an amplification reaction under strand displacement conditions. Under strand displacement conditions, hybridization of the two complementary regions, which may be fully or partially complementary, of the molecular torch is favored, except in the presence of the target sequence, which will bind to the single-stranded region present in the target binding domain and displace all or a portion of the target closing domain. The target binding domain and the target closing domain of a molecular torch include a detectable label or a pair of interacting labels (e.g., luminescent/quencher) positioned so that a different signal is produced when the molecular torch is self-hybridized than when the molecular torch is hybridized to the target sequence, thereby permitting detection of probe:target duplexes in a test sample in the presence of unhybridized molecular torches. Molecular torches and a variety of types of interacting label pairs are disclosed in U.S. Pat. No. 6,534,274, herein incorporated by reference in its entirety.

Another example of a detection probe having self-complementarity is a “molecular beacon.” Molecular beacons include nucleic acid molecules having a target complementary sequence, an affinity pair (or nucleic acid arms) holding the probe in a closed conformation in the absence of a target sequence present in an amplification reaction, and a label pair that interacts when the probe is in a closed conformation. Hybridization of the target sequence and the target complementary sequence separates the members of the affinity pair, thereby shifting the probe to an open conformation. The shift to the open conformation is detectable due to reduced interaction of the label pair, which may be, for example, a fluorophore and a quencher (e.g., DABCYL and EDANS). Molecular beacons are disclosed in U.S. Pat. Nos. 5,925,517 and 6,150,097, herein incorporated by reference in its entirety.

Other self-hybridizing probes are well known to those of ordinary skill in the art. By way of non-limiting example, probe binding pairs having interacting labels, such as those disclosed in U.S. Pat. No. 5,928,862 (herein incorporated by reference in its entirety) might be adapted for use in the present invention. Probe systems used to detect single nucleotide polymorphisms (SNPs) might also be utilized in the present invention. Additional detection systems include “molecular switches,” as disclosed in U.S. Publ. No. 20050042638, herein incorporated by reference in its entirety. Other probes, such as those comprising intercalating dyes and/or fluorochromes, are also useful for detection of amplification products in the present invention. See, e.g., U.S. Pat. No. 5,814,447 (herein incorporated by reference in its entirety).

### C. Protein Detection

The products of the synthetic or endogenous genes, if they differ in amino acid sequence, may be detected and/or differentiated using a variety of protein techniques known to those of ordinary skill in the art, including but not limited to: protein sequencing; and, immunoassays. Where antibodies are employed, the antibodies are generated to selective bind to either the synthetic of endogenous protein relative to the other.

### 1. Sequencing

Illustrative non-limiting examples of protein sequencing techniques include, but are not limited to, mass spectrometry and Edman degradation.

Mass spectrometry can, in principle, sequence any size protein but becomes computationally more difficult as size increases. A protein is digested by an endoprotease, and the resulting solution is passed through a high pressure liquid chromatography column. At the end of this column, the solution is sprayed out of a narrow nozzle charged to a high positive potential into the mass spectrometer. The charge on the droplets causes them to fragment until only single ions remain. The peptides are then fragmented and the mass-charge ratios of the fragments measured. The mass spectrum is analyzed by computer and often compared against a database of previously sequenced proteins in order to determine the sequences of the fragments. The process is then repeated with a different digestion enzyme, and the overlaps in sequences are used to construct a sequence for the protein.

In the Edman degradation reaction, the peptide to be sequenced is adsorbed onto a solid surface (e.g., a glass fiber coated with polybrene). The Edman reagent, phenylisothiocyanate (PTC), is added to the adsorbed peptide, together with a mildly basic buffer solution of 12% trimethylamine, and reacts with the amine group of the N-terminal amino acid. The terminal amino acid derivative can then be selectively detached by the addition of anhydrous acid. The derivative isomerizes to give a substituted phenylthiohydantoin, which can be washed off and identified by chromatography, and the cycle can be repeated. The efficiency of each step is about 98%, which allows about 50 amino acids to be reliably determined.

### 2. Immunoassays

Illustrative non-limiting examples of immunoassays include, but are not limited to: immunoprecipitation; Western blot; ELISA; immunohistochemistry; immunocytochemistry; flow cytometry; and, immuno-PCR. Polyclonal or monoclonal antibodies detectably labeled using various techniques known to those of ordinary skill in the art (e.g., colorimetric, fluorescent, chemiluminescent or radioactive) are suitable for use in the immunoassays.

Immunoprecipitation is the technique of precipitating an antigen out of solution using an antibody specific to that antigen. The process can be used to identify protein complexes present in cell extracts by targeting a protein believed to be in the complex. The complexes are brought out of solution by insoluble antibody-binding proteins isolated initially from bacteria, such as Protein A and Protein G. The antibodies can also be coupled to sepharose beads that can easily be isolated out of solution. After washing, the precipitate can be analyzed using mass spectrometry, Western blotting, or any number of other methods for identifying constituents in the complex.

A Western blot, or immunoblot, is a method to detect protein in a given sample of tissue homogenate or extract. It uses gel electrophoresis to separate denatured proteins by mass. The proteins are then transferred out of the gel and onto a membrane, typically polyvinylidene fluoride (PVDF) or nitrocellulose, where they are probed using antibodies specific to the protein of interest. As a result, researchers can examine the amount of protein in a given sample and compare levels between several groups.

An ELISA, short for Enzyme-Linked ImmunoSorbent Assay, is a biochemical technique to detect the presence of an antibody or an antigen in a sample. It utilizes a minimum of two antibodies, one of which is specific to the antigen and the other of which is coupled to an enzyme. The second antibody will cause a chromogenic or fluorogenic substrate to produce a signal. Variations of ELISA include sandwich ELISA, competitive ELISA, and ELISPOT. Because the ELISA can be performed to evaluate either the presence of antigen or the presence of antibody in a sample, it is a useful tool both for determining serum antibody concentrations and also for detecting the presence of antigen.

Immunohistochemistry and immunocytochemistry refer to the process of localizing proteins in a tissue section or cell, respectively, via the principle of antigens in tissue or cells binding to their respective antibodies. Visualization is enabled by tagging the antibody with color producing or fluorescent tags. Typical examples of color tags include, but are not limited to, horseradish peroxidase and alkaline phosphatase. Typical examples of fluorophore tags include, but are not limited to, fluorescein isothiocyanate (FITC) or phycoerythrin (PE).

Flow cytometry is a technique for counting, examining and sorting microscopic particles suspended in a stream of fluid. It allows simultaneous multiparametric analysis of the physical and/or chemical characteristics of single cells flowing through an optical/electronic detection apparatus. A beam of light (e.g., a laser) of a single frequency or color is directed onto a hydrodynamically focused stream of fluid. A number of detectors are aimed at the point where the stream passes through the light beam; one in line with the light beam (Forward Scatter or FSC) and several perpendicular to it (Side Scatter (SSC) and one or more fluorescent detectors). Each suspended particle passing through the beam scatters the light in some way, and fluorescent chemicals in the particle may be excited into emitting light at a lower frequency than the light source. The combination of scattered and fluorescent light is picked up by the detectors, and by analyzing fluctuations in brightness at each detector, one for each fluorescent emission peak, it is possible to deduce various facts about the physical and chemical structure of each individual particle. FSC correlates with the cell volume and SSC correlates with the density or inner complexity of the particle (e.g., shape of the nucleus, the amount and type of cytoplasmic granules or the membrane roughness).

Immuno-polymerase chain reaction (IPCR) utilizes nucleic acid amplification techniques to increase signal generation in antibody-based immunoassays. Because no protein equivalence of PCR exists, that is, proteins cannot be replicated in the same manner that nucleic acid is replicated during PCR, the only way to increase detection sensitivity is by signal amplification. The target proteins are bound to antibodies that are directly or indirectly conjugated to oligonucleotides. Unbound antibodies are washed away and the remaining bound antibodies have their oligonucleotides amplified. Protein detection occurs via detection of amplified oligonucleotides using standard nucleic acid detection methods, including real-time methods.

### EXPERIMENTAL

The following examples serve to illustrate certain embodiments and aspects of the present invention and are not to be construed as limiting the scope thereof.

### Example 1

### Assessing Expression Levels of Endogenous and Exogenous Nanog

Nanog is a transcription factor involved in the self-renewal of undifferentiated stem cells. Nanog is among the genes commonly used to induce the formation of pluripotent stem cells from somatic cells. The transfection of exogenous Nanog into somatic cells, under proper conditions, induces expression of Nanog from the endogenous version of the same gene. The present invention provides a method to assess the expression levels of the exogenous and endogenous Nanog genes in induced pluripotent stem cells.

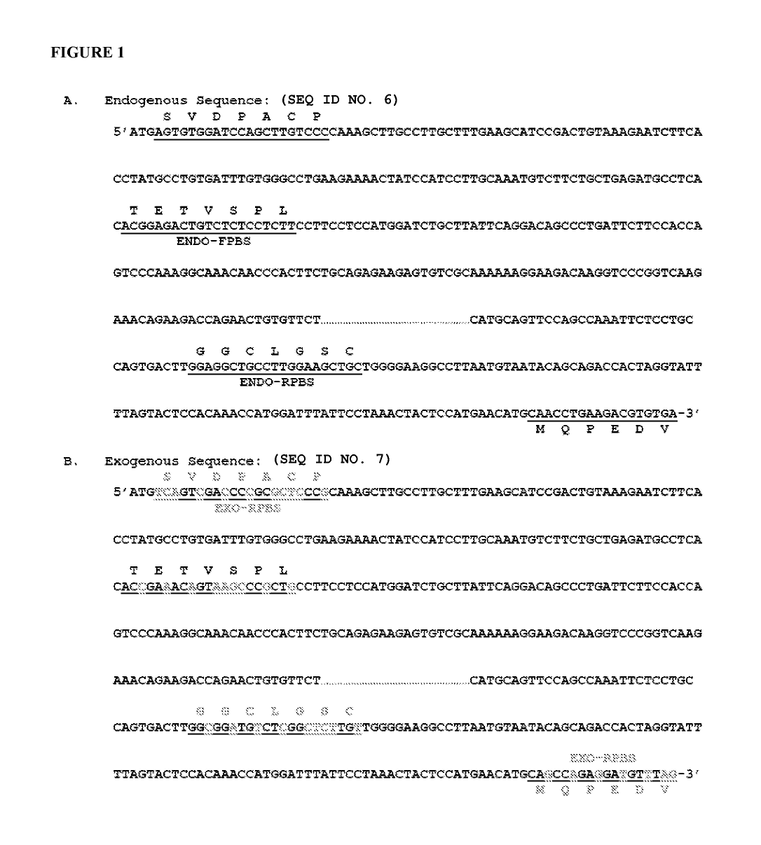

A version of the Nanog gene is synthesized which expresses protein identical to that expressed from the endogenous Nanog gene. The mRNA sequence of the synthetic gene contains nucleotide substitutions over four 21 nucleotide segments across the gene (SEE FIG. 1). The nucleotide substitutions occur at the wobble positions of the mRNA codons, or at other positions which don't affect the amino acid sequence that the gene encodes (SEE FIG. 1). The synthetic gene is transfected into somatic cells (e.g. fibroblasts) according to procedures known to induce formation of pluripotent stem cells.

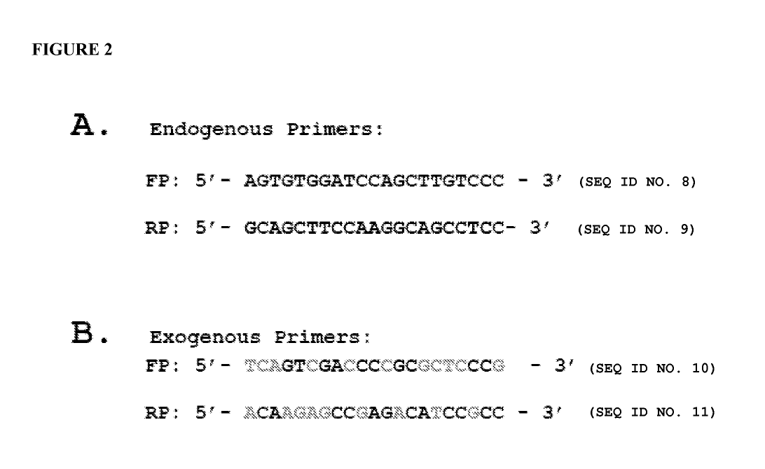

Two sets of primers are designed each complementary to the altered sequences of the endogenous and exogenous Nanog genes, respectively (SEE FIG. 2). The primer sets are capable of hybridizing with either the exogenous or endogenous Nanog gene, but not both.

RNA is extracted from the induced pluripotent stem cells. The extracted RNA is reverse transcribed to produce cDNA across the Nanog genes. The exogenous and endogenous Nanog mRNA is quantified by qPCR (e.g. Plexor qPCR System), using the endogenous and exogenous Nanog primer sets. The locations and sequence of the endogenous and exogenous primer binding sites produce different product amplicons for the enogenous and exogenous genes. By quantifying the mRNA levels of the exogenous and endogenous Nanog mRNA, the present method assesses the expression of endogenous and exogenous Nanog in the induced pluripotent stem cells.

### Example 2

### Synthetic Genes for Assessing iPSCs (SEQ ID NO:1)

The following are examples of synthetic genes for transcription factors which can be used to assess exogenous gene expression in induced pluripotent stem cells. The synthetic genes contain a NcoI site at the 5′ end and an XbaI site at the 3′ end to assist with cloning. Inserted codon changes, without affecting the amino acid coding, are made at the primer annealing sites to allow discrimination of the synthetic sequence from the endogenous transcript. Inserted codon changes are made to eliminate internal NeoI and XbaI sites where necessary to provide a different digestion pattern of amplification products. Primer binding sites are highlighted.

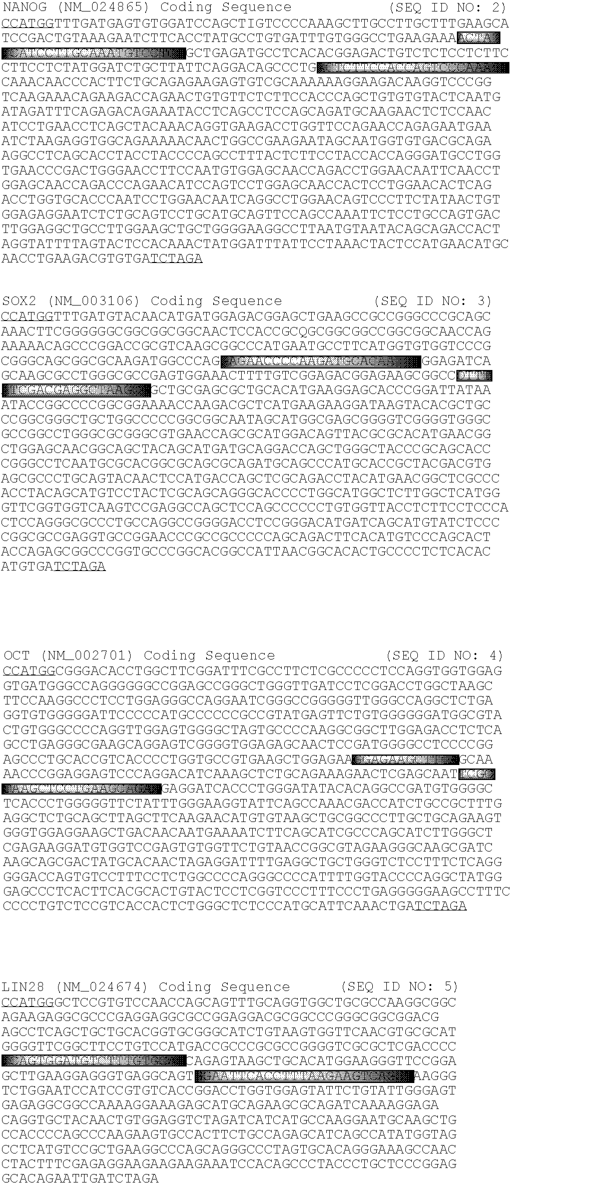

We claim:

1. A method comprising:
a) providing:
i) one or more cells;
ii) an exogenous gene encoding a de-differentiation factor, said exogenous gene differing in sequence from an endogenous version of said gene in said one or more cells;
b) introducing said exogenous gene into said one or more cells to induce de-differentiation of said one or more cells; and
c) detecting the presence of, absence of, or amount of expression of said exogenous gene and said endogenous gene.

2. The method of claim 1, wherein said exogenous gene and said endogenous gene express protein of identical amino acid sequence.

3. The method of claim 1, wherein said discriminating is based on a detectable property in the mRNA of said exogenous gene and said endogenous gene.

4. The method of claim 3, wherein said detectable property is a difference in mRNA sequence of the exogenous gene and said endogenous version of the same gene.

5. The method of claim 4, wherein said difference in mRNA sequence occurs in the wobble position of one or more mRNA codons.

6. The method of claim 4, wherein said difference in mRNA sequence occurs in an untranslated region of said mRNA.

7. The method of claim 3, wherein said discriminating of said detectable property comprises reverse transcription of said mRNA of said exogenous gene and said endogenous gene.

8. The method of claim 3, wherein said discriminating of said detectable property comprises amplification of said exogenous gene and said endogenous gene.

9. The method of claim 3, wherein said discriminating of said detectable property comprises hybridization of one or more probes to said exogenous gene and said endogenous gene.

10. The method of claim 1, wherein said cells are induced pluripotent stem cells.

11. The method of claim 1, wherein said de-differentiation factor is selected from the group consisting of: Oct1, Oct3, Oct4, Oct3/4, Oct6, Sox1, Sox2, Sox3, Sox15, Sox18, Nanog, C-Myc, N-Myc, L-Myc, Klf1, Klf2, Klf4, Klf5, LIN28, and Fbx15.

12. The method of claim 1, further comprising the step of treating the cells with an agent after step b), but prior to step c).

13. A composition comprising:
a) a synthetic, non-native de-differentiation gene having a sequence that differs from a native de-differentiation gene; and
b) a reagent that detects an expression product of said synthetic, non-native de-differentiation gene and differentiates said expression product from an expression of said native de-differentiation gene.

14. The composition of claim 13, wherein said de-differentiation gene is selected from the group consisting of: Oct1, Oct3, Oct4, Oct3/4, Oct6, Sox1, Sox2, Sox3, Sox15, Sox18, Nanog, C-Myc, N-Myc, L-Myc, Klf1, Klf2, Klf4, Klf5, LIN28, and Fbx15.

15. The composition of claim 13, wherein said reagent comprises one or more nucleic acid probes that binds specifically to said expression product from said synthetic, non-native de-differentiation gene relative to said native gene.

16. The composition of claim 13, wherein said reagent comprises one or more primer probes that binds specifically to said expression product from said synthetic, non-native de-differentiation gene relative to said native gene.

17. The composition of claim 13, wherein synthetic gene is present in an expression vector.

18. The composition of claim 17, wherein said expression vector is a viral expression vector.


SEQUENCE LISTING (11 sequences)

SEQ ID NO: 1 - skipped

SEQ ID NO: 2 - DNA, 934 bp, Artificial Sequence
  Synthetic Oligonucleotide
  misc_feature (29)..(29): n represents inosine
  misc_feature (123)..(123): n represents inosine
ccatggtttgatgagtgtggatccagctngtccccaaagcttgccttgctttgaagcatccgactgtaaagaatcttcacctatgcctgtgatttgtgggcctgaagaaaactatccatcctngcaaatgtcctctgctgagatgcctcacacggagactgtctctcctcttccttcctctatggatctgcttattcaggacagccctgactcttccaccagtcccaaaggcaaacaacccacttctgcagagaagagtgtcgcaaaaaaggaagacaaggtcccggtcaagaaacagaagaccagaactgtgttctcttccacccagctgtgtgtactcaatgatagatttcagagacagaaatacctcagcctccagcagatgcaagaactctccaacatcctgaacctcagctacaaacaggtgaagacctggttccagaaccagagaatgaaatctaagaggtggcagaaaaacaactggccgaagaatagcaatggtgtgacgcagaaggcctcagcacctacctaccccagcctttactcttcctaccaccagggatgcctggtgaacccgactgggaaccttccaatgtggagcaaccagacctggaacaattcaacctggagcaaccagacccagaacatccagtcctggagcaaccactcctggaacactcagacctggtgcacccaatcctggaacaatcaggcctggaacagtcccttctataactgtggagaggaatctctgcagtcctgcatgcagttccagccaaattctcctgccagtgacttggaggctgccttggaagctgctggggaaggccttaatgtaatacagcagaccactaggtattttagtactccacaaactatggatttattcctaaactactccatgaacatgcaacctgaagacgtgtgatctaga

SEQ ID NO: 3 - DNA, 969 bp, Artificial Sequence
  Synthetic Oligonucleotide
ccatggtttgatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaattcggagatcagcaagcgcctgggcgccgagtggaaacttttgtcggagacggagaagcggccgtttatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctcatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgtgatctaga

SEQ ID NO: 4 - DNA, 958 bp, Artificial Sequence
  Synthetic Oligonucleotide
ccatggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagcttgagcaaaacccggaggagtcccaggacatcaaagctctgcagaaagaactcgagcaattcgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcagcatcgcccagcatcttgggctcgagaaggatgtggtccgagtgtggttctgtaaccggcgtagaagggcaagcgatcaagcagcgactatgcacaactagaggattttgaggctgctgggtctcctttctcagggggaccagtgtcctttcctctggccccagggccccattttggtaccccaggctatgggagccctcacttcacgcactgtactcctcggtccctttccctgagggggaagcctttccccctgtctccgtcaccactctgggctctcccatgcattcaaactgatctaga

SEQ ID NO: 5 - DNA, 638 bp, Artificial Sequence
  Synthetic Oligonucleotide
ccatgggctccgtgtccaaccagcagtttgcaggtggctgcgccaaggcggcagaagaggcgcccgaggaggcgccggaggacgcggcccgggcggcggacgagcctcagctgctgcacggtgcgggcatctgtaagtggttcaacgtgcgcatggggttcggcttcctgtccatgaccgcccgcgccggggtcgcgctcgaccccccagtggatgtctttgtgcatcagagtaagctgcacatggaagggttccggagcttgaaggagggtgaggcagtggaattcacctttaagaagtcagccaagggtctggaatccatccgtgtcaccggacctggtggagtattctgtattgggagtgagaggcggccaaaaggaaagagcatgcagaagcgcagatcaaaaggagacaggtgctacaactgtggaggtctagatcatcatgccaaggaatgcaagctgccaccccagcccaagaagtgccacttctgccagagcatcagccatatggtagcctcatgtccgctgaaggcccagcagggccctagtgcacagggaaagccaacctactttcgagaggaagaagaagaaatccacagccctaccctgctcccggaggcacagaattgatctaga

SEQ ID NO: 6 - DNA, 472 bp, Artificial Sequence
  Synthetic Oligonucleotide
atgagtgtggatccagcttgtccccaaagcttgccttgctttgaagcatccgactgtaaagaatcttcacctatgcctgtgatttgtgggcctgaagaaaactatccatccttgcaaatgtcttctgctgagatgcctcacacggagactgtctctcctcttccttcctccatggatctgcttattcaggacagccctgattcttccaccagtcccaaaggcaaacaaccccttctgcagagaagagtgtcgcaaaaaaggaagacaaggtcccggtcaagaaacagaagaccagaactgtgttctcatgcagttccagccaaattctcctgccagtgacttggaggctgccttggaagctgctggggaaggccttaatgtaatacagcagaccactaggtattttagtactccacaaaccatggatttattcctaaactactccatgaacatgcaacctgaagacgtgtga

SEQ ID NO: 7 - DNA, 473 bp, Artificial Sequence
  Synthetic Oligonucleotide
atgtcagtcgaccccgcgctcccgcaaagcttgccttgctttgaagcatccgactgtaaagaatcttcacctatgcctgtgatttgtgggcctgaagaaaactatccatccttgcaaatgtcttctgctgagatgcctcacaccgaaacagtaagcccgctgccttcctccatggatctgcttattcaggacagccctgattcttccaccagtcccaaaggcaaacaacccacttctgcagagaagagtgtcgcaaaaaaggaagacaaggtcccggtcaagaaacagaagaccagaactgtgttctcatgcagttccagccaaattctcctgccagtgacttggcggatgtctcggctcttgttggggaaggccttaatgtaatacagcagaccactaggtattttagtactccacaaaccatggatttattcctaaactactccatgaacatgcagccagaggatgtttag

SEQ ID NO: 8 - DNA, 21 bp, Artificial Sequence
  Synthetic Oligonucleotide
agtgtggatccagcttgtccc

SEQ ID NO: 9 - DNA, 21 bp, Artificial Sequence
  Synthetic Oligonucleotide
gcagcttccaaggcagcctcc

SEQ ID NO: 10 - DNA, 21 bp, Artificial Sequence
  Synthetic Oligonucleotide
tcagtcgaccccgcgctcccg

SEQ ID NO: 11 - DNA, 21 bp, Artificial Sequence
  Synthetic Oligonucleotide
acaagagccgagacatccgcc